(19) United States
(12) Patent Application Publication
(10) Pub. No.: US 2002/0001661 A1
(43) Pub. Date: Jan. 3, 2002

(54) Low calorie fat compositions

(76) Inventor: Damodara Gopal, Cleveland, OH (US)

Correspondence Address: Senniger, Powers, Leavitt & Roedel, One Metropolitan Square, 16th Floor, St. Louis, MO, 63102, US

(21) Appl. No.: 09/785,807

(22) Filed: Feb. 16, 2001

Related U.S. Application Data
(60) Provisional application No. 60/183,827, filed on Feb. 17, 2000.

(51) Int. Cl. A23D 9/00

(52) U.S. Cl. 426/601

(57) ABSTRACT

By this invention, low calorie fat compositions comprising the use of hydroxy triglycerides are provided. Of particular interest is the use of triglyceride compositions having a at least one hydroxy fatty acid. Preferably, the hydroxy fatty acid has a melting temperature above 40° C. The hydroxy triglyceride compositions find use as low calorie fat ingredients in food compositions. The low calorie fat compositions of the present invention demonstrate a caloric value of less than 5.0 kcal/g. Such low calorie fat compositions find use in the preparation of food compositions wherein at least a portion of the fat ingredients have been replaced with a hydroxy triglyceride composition of the present invention.

### CROSS REFERENCE TO RELATED APPLICATIONS

This application claims the benefit of priority to U.S. Provisional Application Ser. No. 60/183,827, which disclosure is herein incorporated by reference.

### INTRODUCTION

1. Technical Field

The present invention relates to low calorie fat compositions. More specifically, the present invention relates to triglyceride compositions containing hydroxy fatty acids for use in low calorie fat compositions, and in particular, the use of such triglyceride compositions in low calorie fat-containing food compositions.

2. Background

One of the most common metabolic conditions today is obesity. Of the various reasons for the condition, ingestion of a greater number of calories than are needed is a primary factor. The typical diet comprises about 40% of the total calories from fat, however, dietary guidelines call for reducing fat intake to less than 30% of the total calorie intake.

Fat contributes much to the palatability and flavor of food as most flavor compounds are fat soluble. Furthermore, fats contribute to the satiety value of foods since fatty foods are slower to digest than foods containing protein and carbohydrates. In addition, fats are carriers of fat soluble vitamins, such as A, D, E, and K, and essential fatty acids, which have been shown to be important in growth and maintenance of many body functions.

One of the major problems with dietary fats is that it is highly calorically dense, about 9 calories per gram, compared to about 4 calories per gram for proteins and carbohydrates. Furthermore, dietary fats can be readily stored by the body when consumed in excess, contributing to the obesity condition. Hence, major research efforts have focused on ways to produce food substances that provide the same functionality and organoleptic properties as fats, but with fewer calories. Recently, research efforts have focused on the synthesis of low calorie fats (U.S. Pat. No. 3,579,548; Hamm, (1984) J. Food Sci. 49:419-428; EP 0910955; U.S. Pat. No. 3,637,774, and U.S. Pat. No. 4,582,715), and several products are currently on the market. Synthetic fats have been created and are currently being marketed. Unfortunately, many consumers are concerned with the gastrointestinal side effects associated with the synthetic fats, as well as with vitamin sequestration.

There is a need in the art for low calorie fat compounds which have the preferable organoleptic properties of normal triglyceride fats, are readily attainable or produced, and do not have adverse side effects.

### SUMMARY OF THE INVENTION

By this invention, low calorie triglyceride compositions and food compositions incorporating them are provided. The triglyceride compositions described herein provide a reduced caloric value compared to normal corn oil. In addition, the triglyceride compositions of the present invention have the preferable organoleptic properties of normal triglycerides.

Thus, a first aspect of the present invention provides low calorie fat compositions comprising triglycerides having at least one hydroxy fatty acid (referred to herein as hydroxy triglycerides). The hydroxy triglyceride compositions of the present invention find particular use in the preparation of various food compositions as a low calorie fat.

An important aspect of the present invention provides a low calorie triglyceride composition having desirable organoleptic properties and functional characteristics useful in a wide variety of food applications. Furthermore, the low calorie triglycerides of the present invention are hydrolyzed by pancreatic lipases similarly to normal triglycerides, however, the hydrolyzed hydroxy fatty acids are poorly absorbed in the intestines.

The hydroxy fatty acids for use in the triglyceride compositions of the present invention have a melting temperature above about 40° C., preferably, the hydroxy fatty acids have a melting temperature between about 40° C. and about 115° C., more preferably between about 40° C. and about 90° C.

The hydroxy fatty acids for use in the hydroxy triglyceride compositions can be either saturated or unsaturated. Preferably the saturated hydroxy fatty acids can be short, medium or long chain hydroxy fatty acids, and the unsaturated hydroxy fatty acids are long chain hydroxy fatty acids. The other positions of the triglyceride molecule are occupied by aliphatic groups, hydrogen, or additional hydroxy fatty acids.

In another aspect of the present invention, low calorie fat compositions of particular interest in the present invention comprise a triglyceride having the formula:

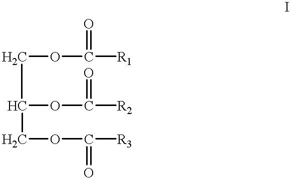

where at least one of the R₁, R₂ and R₃ group has the following formula II or III

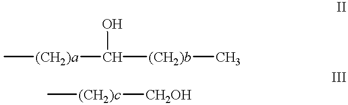

wherein a is 0 to 22, b is 0 to 22, and c is 0 to 22. The remaining fatty acids comprise short chain fatty acids (C2 to C5) or medium chain fatty acids (C6 to C12) or other hydroxy fatty acids.

These hydroxy triglyceride compositions can be used either alone or in combination with additional vegetable oils. Furthermore, the compositions can be hydrolyzed to the high melting and poorly absorbed long chain hydroxy fatty acids.

The hydroxy triglyceride compositions of the present invention particularly find use in low calorie food compositions.

### DETAILED DESCRIPTION OF THE INVENTION

In accordance with the subject invention, compositions and methods related to low calorie fats are provided. In particular, the present invention provides triglyceride compositions having at least one hydroxy fatty acid and methods of use as low calorie fats in food compositions.

The hydroxy triglycerides of the present invention, and fat-containing food compositions containing these compounds, have desirable physical properties and palatability compared to ordinary triglyceride fats and compositions containing same. However, these hydroxy triglycerides have substantially lower effective caloric value because even though they are readily hydrolyzable by lipase, they are poorly absorbed in the intestinal tract and are reduced in caloric availability compared to ordinary triglyceride fat. The hydroxy triglycerides and the food compositions containing these compounds which are low in available calories are referred to herein as “low calorie”.

The hydroxy triglycerides of particular interest in the present invention comprise a glycerol backbone, esterified to at least one hydroxy fatty acid, preferably a hydroxy fatty acid, at the sn-1, sn-2, and/or sn-3 positions of the triglyceride. The remainder of the positions of the glycerol molecule are occupied by aliphatic groups, straight chain or branched, hydrogen, or additional hydroxy fatty acids.

Thus, a first aspect of the present invention provides triglyceride compositions comprising at least one hydroxy fatty acid. Such compositions are referred to herein as hydroxy triglycerides.

Another aspect of the present invention provides a triglyceride composition having the formula:

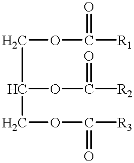

where at least one of the R1, R2 and R3 groups is a hydroxy fatty acid which can be represented by the formula II or III.

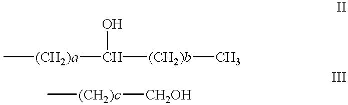

wherein a, b and c are 0 to 30, preferably 0 to 26, more preferably 0 to 22. The hydroxy fatty acid of formulas II and III can be a saturated or unsaturated fatty acids. Saturated hydroxy fatty acids for use in the triglycerides of formula I comprise those from 0 to 30 carbons, preferably 0 to 26, more preferably 0 to 22 carbons. The unsaturated hydroxy fatty acids for use in the triglycerides of formula I comprise those between 20 and 30 carbons, preferably 20 to 26 carbons. The remaining R positions can be esterified with aliphatic hydrocarbons, hydrogen, or additional hydroxy fatty acids of the formulas II and III.

Any hydroxy fatty acid can be used in the low calorie fat triglycerides of the present invention. Preferred hydroxy fatty acids for use in the compositions of the present invention include those that have a melting temperature above about 40° C. In particular, the hydroxy fatty acids have a melting temperature between about 40° C. and about 120° C., preferably between about 40° C. and about 115° C., more preferably, between about 50° C. and about 110° C.

Furthermore, the hydroxy fatty acids for use in the triglyceride compositions of the present invention can be saturated or unsaturated acyl hydrocarbons and contain between about 1 and about 30 carbons. Particularly, saturated hydroxy fatty acids contain between about 1 and about 30 carbons, more preferably between about 1 and about 26 carbons, most especially preferable between about 1 and about 22 carbons. Furthermore, the hydroxy group can be substituted at any position of the fatty acid chain, and can include more than one hydroxy group along the fatty acid chain. Unsaturated hydroxy fatty acids for use in the hydroxy triglyceride compositions of the present invention are preferably long chain unsaturated hydroxy fatty acids having at least about 18 carbons to about 30 carbons.

Examples of hydroxy fatty acids for use in the compositions of the present invention include, but not limited to, saturated natural hydroxy fatty acids juniperic acid, butolic acid, ipurolic acid, dihydroxy stearic acids, dihydroxy palmitic acids, hydroxynervonic acid, alpha-kamlolenic acid, beta-kamlolenic acid, or hydrogenated natural hydroxy acids ricinoleic, isoricinoleic, densipolic, lesquerolic, auricolic, ximenynolic, isanolic, dimorphecolic, coriolic, or synthetic saturated hydroxy faty acids 9-hydroxy stearic acid, 10-hydroxy stearic acid. The hyxroxy acids also include saturated dihydroxy and polyhydroxy acids like ipurolic, ustilic, aleuritic, 12,13-dihydroxypalmitic and 12,13-dihydroxystearic, 9,10-dihydroxystearic acids.

Additional fatty acids for use in the hydroxy triglyceride compositions of the present invention can be derived from straight chain fatty acids and/or branched chain fatty acids of chains 2 to 14 carbons long. The term fatty acids encompasses synthetic and natural organic carboxylic acids having the formula represented as RCOOH. Examples of this type of saturated fatty acids include, but are not limited to, acetic, propionic, butyric, caproic, caprylic, capric, lauric, myristic acids. The triglycerides derived from these compounds can have one to three of the hydroxy acids. These triglycedies can be derived from random mixtures of hydroxy fatty acids and non-hydroxy fatty acids.

The hydroxy triglyceride compositions of the present invention provide a triglyceride oil source having reduced caloric availability compared to normal triglyceride fats and oils . The hydroxy triglyceride compositions of the present invention preferably provide less than about 5.5 kcal/g, more preferably less than 5.1 kcal/g, especially preferred less than about 4.7 kcal/g, most especially preferred, less than about 4.2 kcal/g. Caloric availability can be determined using a variety of methods such as those described, for example, by Finley, et al. ((1994) J. Agric. Food Chem. 42:489-494) and Peters et al. (1993) J. Amer. College of Toxic. 10:357-367.

In addition, other analysis can be helpful in determination of digestibility and or absorbability of hydroxy triglyceride compounds. For example, resistance to hydrolysis by pancreatic enzymes can determine the digestibility of the hydroxy triglyceride molecules in vitro. Preferably, the hydroxy triglyceride compositions of the present invention are readily hydrolyzed by pancreatic lipases, similar to normal corn triglycerides. Methods for in vitro pancreatic lipase analysis are known in the art and are described for example by Volpenhein (U.S. Pat. No. 4,582,715).

The hydroxy triglyceride compositions for use as low calorie fat compositions of the present invention can be obtained from any source, including natural and synthetic sources. Also included as a source of the hydroxy triglycerides are genetically engineered sources, such as yeasts, bacterial, plants and the like.

Hydroxy fatty acids for use in the hydroxy triglycerides can be produced using any method available in the art from a wide variety of starting materials. For example, hydroxy fatty acids, or their esters, can be used as starting material for the production of the hydroxy triglyceride compositions of the present invention. Alternatively, natural hydroxy triglyceride sources can also be employed, or unsaturated fatty acids can be hydroxylated, or substituted fatty acids can also be used.

Thus, the hydroxy triglyceride compositions of the present invention provide low calorie fat compositions which find use in the preparation of a wide variety of food applications.

The low calorie fat compositions of the present invention can be used as a partial or total replacement for normal fats in any fat-containing food product comprising fat and nonfat ingredients to provide reduced calorie benefits. In order to obtain a significant reduction in calories, at least about 5%, and preferably at least about 20%, of the total fat in the food product comprises the low calorie fat composition of the present invention. Alternatively, 100% replacement of normal fats with the low calorie triglyceride compositions of the present invention provides a highly desirable food composition. In addition, the low calorie triglyceride compositions can be blended with other low calorie fats, fat replacers or fat mimetics.

Thus, the hydroxy triglyceride compositions of the present invention can be used in a variety of applications. Of particular interest in the present invention is the use of the low calorie triglyceride compositions in various food applications. Of most particular interest is the use in low calorie food applications.

Thus, the hydroxy triglyceride compositions of the present invention find use in the preparation of foods, food products, processed foods, food ingredients, food additive compositions, or dietary supplements that contain oils and/or fats. Examples of such uses include but are not limited to margarines, butters, shortenings, dressings, spreads, frying oils, mayonnaises, and vitamin/mineral supplements. Additional examples include, but are not limited to toppings, dairy products such as cheese and processed cheese, processed meat and meat mimetics, pastas, cereals, sauces, desserts including frozen and shelf stable desserts, dips, chips, baked goods, pastries, cookies, snack bars, confections, chocolates, beverages, unextracted seed, and unextracted seed that has been ground, cracked, milled, rolled, extruded, pelleted, defatted, dehydrated, or otherwise processed, but which still contains the oils, etc., disclosed herein.

Furthermore, hydroxytriglyceride compositions incorporating medium chain fatty acids can also be used in ketogenic food compositions for use in ketogenic diets.

The invention now being generally described, it will be more readily understood by reference to the following examples which are included for purposes of illustration only and are not intended to limit the present invention.

### EXAMPLES

Unless otherwise indicated, all parts and percentages are by weight.

### Example 1: Preparation of Hydroxy Triglycerides

1A. Preparation of Glycerol 12-hydroxystearic-diproppionate

This process produces a random mixtures of triglycerides containing 12-hydroxy stearic acid and propionic acid the ratio of which can be varied. For example, using one mole hydrogenated castor oil with five moles of tripropionin under inter-esterification conditions a product containing one 12-hydroxy stearic acid and two propionic acid are prepared.

Step 1: Preparation of glycerol tri-12-tetrahydropyranyloxy stearate

Hydrogenated castor oil (1.5 Kg) and dihydropyran (4.36 Kg) were reacted in the presence of catalytic amount of pyridinium p-toluene sulfonate (PPTS, 3 g) for 3 hr at 86° C. The excess dihydropyran was removed followed by the dissolution of the crude product in hexanes (6 L). The solution was washed with water, and bicarbonate solution. The dried solution was concentrated to get the THP protected hydrogenated castor oil (1.8 Kg).

Step 2: Preparation of glycerol 12-tetrahydropyranyloxystearic-dipropionate

Inter-esterification of glycerol tri-12-tetrahydropyranyloxy stearate and tripropionin

The THP protected hydrogenated castor oil (300 g) and tripropionin (252 g) were inter-esterified using sodium methoxide (2.4 g). The reaction was carried out at 90-100° C. for 3 hr. The product containing excess tripropionin was isolated using filterol (523 g).

Step 3: Preparation of glycerol 12-hydroxystearic-diproppionate

The protection THP group (523 g) was removed using ethanol (3.5 L)and PPTS (4 g) at 60° C. for 3 hr. Water (300 ml) was added and the solvent was removed. The crude product was dissolved in hexanes (3 L) and washed with water (3×1 L) and bicarbonate solution. The solution was dried (MgSO₄) and concentrated. The excess tripropionin in the final product was removed using a short column distillation followed by deodorization to yield (365 g).

This product can also be prepared from castor oil using the same procedure and hydrogenating the final product or any of the intermediate. Products having various solid fat index can be prepared by using different ratio of starting materials and following the same procedure.

1B. Preparation of Glycerol (16-hydroxyhexadecanoic) dipropionate

Step: 1 Preparation of methyl 16-hydroxyhexadecanoate

A mixture of 16-hydroxyhexadecanoaic acid (50 g), dimethoxypropanes (20 g), were refluxed in methanol for 6 hours in the presence of catalytic amount of p-toluenesulfonic acid (0.5 g). The solvent was removed after neutralizing the acid catalyst with 20 ml of saturated sodium bicarbonate and the product was extracted in dichloromethane (200 ml). The solution was washed with water, cold saturated sodium bicarbonate solution and dried (MgSO₄). Evaporation of the solvent gave methyl 16-hydroxyhexadecanoate (47 g).

Step: 2 Preparation of methyl 16-tetrahydropyranyloxyhexadecanoate

Methyl 16-hydroxyhexadecanoate (45 g) was reacted with dihydropyran (52.7 g) in dichloromethane using PPTS as catalyst. After six hours the solution was washed with sodium bicarbonate solution, water and dried (MgSO₄). The removal of the solvent under reduced pressure gave the product (51 g).

Step: 3 Preparation of glycerol (16-tetrahydropyranyloxyhexadecanoic) dipropionate

Methyl 16-tetrahydropyranyloxyhexadecanoate (50 g) was inter-esterified with tripropionin (214 g) using sodium methoxide as catalyst. The reaction was run for 4 hours at 100° C. under a vacuum of 10 mm. The product was isolated by passing through filterol. (Yield 230 g)

Step: 4 Preparation of glycerol (16-hydroxyhexadecanoic) dipropionate

Glycerol 16-tetrahydropyranyloxyhexadecanoic dipropionate (200 g) was dissolved in ethanol and heated to 60° C. in the presence of PPTS (2 g). After 6 hours the excess ethanol was removed at room temperature and the crude product was extracted in hexanes (1 L). The solution was washed with water (2×1 L) and dried (MgSO₄). Evaporation of the solvent gave 178 g of the product. The excess tripropionin was removed by passing through short column distillation.

1C. Preparation of glycerol (13/14-hydroxydocosanoic dibutyrate

Step 1: Preparation glycerol 13-docosenoic dibutyrate

Erucic acid methyl ester (105.6 g) and tributyrin (725 g) were inter-esterified using sodium methoxide. The reaction was carried out at 90-100° C. for 3 hr under reduced pressure. The product was isolated using filterol (750 g). The excess tributyrin in the product was removed by short-column distillation under reduced pressure.

Step 2: Preparation of glycerol (13/14-hydroxydocosanoic) dibutyrate

Glycerol (13-docosenoic) dibutyrate (55 g) was dissolved in dry THF (1 L) and reacted with BH₃-THF (1 M, 35 ml). An equivalent amount of a solution of sodium hydroxide was added followed by the addition of hydrogen peroxide. After stirring for two hours the excess THF was removed and the product was extracted with dichloromethane. The solution was dried (MgSO₄), concentrated and the product was deodorized (45 g).

### Example 2: Caloric Availability

Caloric availability of the compounds are evaluated by the two week feeding study of young male Sprague-Dawley rats weighing approximately 50 to 60 gm as described by Finley, et al. ((1994) J. Agric. Food Chem. 42:489-494). Feeding studies were conducted for each compound at 15% fat level using a modified AIN-76 diet. The modified diet has the following compositions.

| Ingredient | g/kg diet |
|---|---|
| Casein | 267.0 |
| DL-Methionine | 6.0 |
| Sucrose | 385.0 |
| Corn Starch | 150.0 |
| Cellulose | 50.0 |
| Corn oil | 50.0 |
| AIN-76 Mineral Mix | 70.0 |
| AIN-76A Vitamin Mix | 10.0 |
| AIN-93 Vitamin Mix | 10.0 |
| Choline Bitartarate | 2.0 |

Weight gains are monitored at days 0, 3, 7, 10 and 14.

Caloric availability of glycerol 12-hydroxystearic-dipropionate was estimated to be 4.2 kcal/g based on the weight gain in the two week feeding study. Caloric value of corn oil was determined to be about 9 kcal/g based on weight gain during the two week feeding study. A triglyceride having one 12-hydroxystearic acid and two medium chain fatty acids (C8, C10 and C12) was estimated to be 7.0 kcal/g based on the weight gain in the two week feeding study.

The above results demonstrate that triglycerides containing at least one hydroxy fatty acid are lower in caloric value than normal triglycerides. Furthermore, such hydroxytriglycerides are useful in the preparation of low calorie fat compositions for use in the production of low calorie food compositions.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claim.

What is claimed is:

1. A low calorie fat comprising a triglyceride having at least one hydroxy fatty acid.

2. The low calorie fat according to claim 1, wherein said hydroxy fatty acid has a melting point of greater than about 40° C.

3. The low calorie fat according to claim 1, wherein said hydroxy fatty acid has a melting point between about 40° C. and about 120° C.

4. The low calorie fat according to claim 1, wherein said hydroxy fatty acid has a melting point between about 50° C. and about 110° C.

5. The low calorie fat according to claim 1, wherein said hydroxy fatty acid is saturated.

6. The low calorie fat according to claim 5, wherein said hydroxy fatty acid is selected from the group consisting of hydroxystearic acid, hydroxyhexadecanoic acid, hydroxyicosanoic acid, and hydroxydocosanoic acid.

7. The low calorie fat according to claim 1, wherein said triglyceride has a caloric value of less than about 5.5 kcal per gram.

8. The low calorie fat according to claim 1, wherein said triglyceride has a caloric value of less than about 5.0 kcal per gram.

9. The low calorie fat according to claim 1, wherein said triglyceride has a caloric value of less than about 4.5 kcal per gram.

10. A fat ingredient having the formula (I): 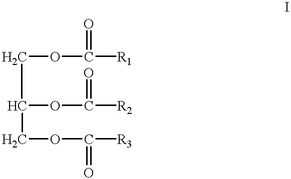
wherein at least one of said R₁, R₂ and R₃ group having the formula (II): 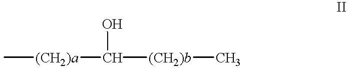
wherein a is 0 to 26 and b is 0 to 26; the remainder of said R₁, R₂ and R₃ are selected from the group consisting of hydrogen, C₁ to C₁₂, and hydroxy fatty acids of formula II.

11. The fat ingredient according to claim 10, wherein a is 14 to 26.

12. The fat ingredient according to claim 10, wherein a is 14 to 22.

13. The fat ingredient according to claim 10, wherein b is 14 to 26.

14. The fat ingredient according to claim 10, wherein b is 14 to 22.

15. The fat ingredient according to claim 10, wherein said fat ingredient has a caloric value of less than about 5.5 kilocalories per gram.

16. The fat ingredient according to claim 10, wherein said fat ingredient has a caloric value of less than about 5.0 kilocalories per gram.

17. The fat ingredient according to claim 10, wherein said fat ingredient has a caloric value of less than about 4.5 kilocalories per gram.

18. A food composition comprising fat ingredients and nonfat ingredients, wherein at least a portion of the fat ingredients comprise the fat ingredient of claim 10.

19. A fat ingredient having the formula (I): 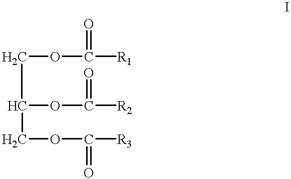
wherein at least one of said R₁, R₂ and R₃ group having the formula (III): 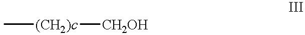
wherein c is 0 to 26; the remainder of said R₁, R₂ and R₃ are selected from the group consisting of hydrogen, C₁ to C₁₂, and hydroxy fatty acids of formula II.

20. The fat ingredient according to claim 19, wherein c is 10 to 26.

21. The fat ingredient according to claim 19, wherein c is 14 to 22.

22. The fat ingredient according to claim 19, wherein c is 18.

23. The fat ingredient according to claim 19, wherein said fat ingredient has a caloric value of less than about 5.5 kilocalories per gram.

24. The fat ingredient according to claim 19, wherein said fat ingredient has a caloric value of less than about 5.0 kilocalories per gram.

25. The fat ingredient according to claim 19, wherein said fat ingredient has a caloric value of less than about 4.5 kilocalories per gram.

26. A food composition comprising fat ingredients and nonfat ingredients, wherein at least a portion of the fat ingredients comprise the fat ingredient of claim 19.

27. A fat ingredient having the formula (I): 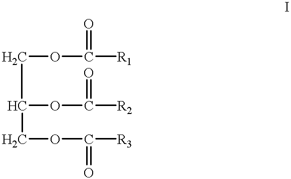
wherein at least one of said R₁, R₂ and R₃ is selected from the group consisting of, formula (II): 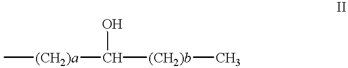
and formula (III) 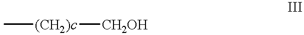
wherein a is 0 to 26, b is 0 to 26, and c is 0 to 26; the remainder of said R₁, R₂ and R₃ are selected from the group consisting of hydrogen, C₁ to C₁₂, hydroxy fatty acids of formula II and formula III.

28. The fat ingredient according to claim 27, wherein a is 10 to 26.

29. The fat ingredient according to claim 27, wherein a is 14 to 22.

30. The fat ingredient according to claim 27, wherein b is 10 to 26.

31. The fat ingredient according to claim 27, wherein b is 14 to 22.

32. The fat ingredient according to claim 27, wherein c is 10 to 26.

33. The fat ingredient according to claim 27, wherein c is 14 to 22.

34. The fat ingredient according to claim 27, wherein said fat ingredient has a caloric value of less than about 5.5 kilocalories per gram.

35. The fat ingredient according to claim 27, wherein said fat ingredient has a caloric value of less than about 5.0 kilocalories per gram.

36. The fat ingredient according to claim 27, wherein said fat ingredient has a caloric value of less than about 4.5 kilocalories per gram.

37. A food composition comprising fat ingredients and nonfat ingredients, wherein at least a portion of the fat ingredients comprise the fat ingredient of claim 27.

38. A low calorie fat containing food composition, which comprises
(a) non-fat ingredients; and
(b) fat ingredients,
wherein said fat ingredients comprise from about 5 to 95% by weight of said fat ingredients of claims 10, 18 and 26.
